(19) United States
(12) Patent Application Publication
(10) Pub. No.: US 2019/0269413 A1
(43) Pub. Date: Sep. 5, 2019

(54) APPARATUS AND METHOD OF MONOFILAMENT IMPLANT DELIVERY IN A BODY VESSEL OF A PATIENT

(71) Applicant: Javelin Medical Ltd., Rehovot (IL)

(72) Inventors: Ofer YODFAT, Modi'in (IL); Guy SHINAR, Ramat Gan (IL)

(21) Appl. No.: 16/153,564

(22) Filed: Oct. 5, 2018

Related U.S. Application Data
(63) Continuation of application No. 14/753,703, filed on Jun. 29, 2015, now abandoned.
Application No. 14/753,703 is a continuation-in-part of application No. 14/552,890, filed on Nov. 25, 2014, now Pat. No. 9,220,588.
Application No. 14/552,890 is a continuation of application No. PCT/IB2013/001336, filed on May 30, 2013.
(60) Provisional application No. 61/653,676, filed on May 31, 2012.
(60) Provisional application No. 61/693,979, filed on Aug. 28, 2012.
(60) Provisional application No. 61/746,423, filed on Dec. 27, 2012.
(60) Provisional application No. 61/754,264, filed on Jan. 18, 2013.

(51) Int. Cl. A61B 17/12; A61B 17/04; A61F 2/88; A61F 2/966; A61F 2/01

(52) U.S. Cl. CPC A61B 17/12145; A61B 17/0401; A61B 17/0487; A61B 17/12031; A61B 17/12036; A61B 2017/00004; A61F 2/88; A61F 2/966; A61B 17/12113; A61F 2/01; A61B 17/0467; A61B 17/12109

(57) ABSTRACT

Embodiments of the present disclosure are directed to methods, systems and devices for implanting a spatially bent and/or twisted implant in a patient. In some embodiments, such a method includes providing a mono-filament implant configured to assume an undeployed, substantially linear state or linear-like state and a spatially bent and/or twisted deployed state, with the plant having a proximal and a distal end. In the undeployed state, the implant includes a shape which corresponds to that of a lumen of a hollow needle which may be used to deliver the implant. The method may also include creating a puncture in a vessel of the patient and positioning said distal end of the needle (and thus, the implant) in the vessel through the puncture. The method may further include converting the implant from the undeployed state to the deployed state such that the proximal end of the implant is proximate said puncture.

### RELATED APPLICATIONS

This application claims priority to U.S. Provisional Patent Application No. 61/746,423, filed Dec. 27, 2012, entitled “Apparatus and Method of Monofilament Implant Delivery in a Body Vessel of a Patient”, the disclosure of which is incorporated herein by reference in its entirety.

### FIELD OF THE DISCLOSURE

The field of the disclosure relates generally to medical implants, and more particularly, to mono-filament implants configured to expand into a spatially twisted arrangement.

### BACKGROUND OF THE DISCLOSURE

Expandable implantable devices are often used for opening and closing of passageways or orifices within the vascular, urinary, or gastrointestinal (GI) systems. Examples include vascular and GI stents for opening occlusions, left atrial appendage (LAA) and patent foramen ovale (PFO) occluding devices, and others. The implantable devices typically comprise a scaffold that is introduced in a collapsed state and is then expanded to a desired configuration at the target organ.

Vascular implants are typically inserted under local anesthesia through peripheral arteries or veins in a patient's leg, arm, or neck—a procedure that is known as endovascular catheterization. The device is collapsed and preloaded into a delivery catheter, advanced trans-luminally to the desired implantation site, and deployed in an expanded configuration. The delivery catheter is usually introduced through a guiding catheter that provides navigation capabilities. The minimal diameter of a guiding catheter is 5-6 French (1.8-2 mm). Therefore, the access puncture (hole) in the skin and vessels cannot be smaller than approximately 2 mm.

Examples for diseases that are currently being treated by an endovascular approach are:
Varicose veins (VV), which are veins that have become enlarged and tortuous causing aching, swelling, itchiness, and skin eczema. Current treatments are targeted to occlude the veins and include surgery (stripping) and non-surgical treatment: sclerotherapy and thermal ablation. These procedures require widespread surface anesthesia in addition to the above mentioned complications of endovascular catheterization.
Left atrial appendage (LAA), which is a muscular pouch connected to the left atrium of the heart. In atrial fibrillation, blood clots originating from the LAA may dislodge and lead to stroke. LAA occlusion devices are a treatment modality for preventing stroke in atrial fibrillation patients. They include expandable scaffolds for occlusion of the LAA orifice. Their associated endovascular catheterization procedure requires puncturing the atrial septum. The procedure is time consuming, and has specific complications such as pericardial effusion, device migration, and others.
Atrial septal defect (ASD), which is a form of congenital heart defect that enables blood flow between the left and right atria. Treatment options include surgery—closing the defect with a patch under direct visualization, or endovascular catheterization. This requires inserting an atrial septal occluder (ASO) device that consists of two self-expandable round discs connected to each other with a short waist.
Stenotic segments in arterial vasculature. The stenotic portions of arteries can be bypassed in a surgical operation, or can be endovascularly dilated by balloon angioplasty followed by stent insertion.
Occlusions and strictures in the GI tract. These can be dilated by an endoluminal approach that includes navigation inside the GI lumen and insertion of a stent to maintain lumen patency.

Endovascular catheterization is performed in a special catheterization laboratory under X-ray guidance. The procedure has many complications including bleeding and vessel rupture at the insertion and remote sites, procedure related embolization, contrast agent toxicity, radiation exposure, and others. In addition, the procedure is costly, time consuming, and requires highly skilled personnel and sophisticated equipment.

There is therefore a need for a method for insertion of an implantable device without a catheterization lab, sophisticated equipment, and highly skilled personal.

There is also a need to provide a method for insertion of an implantable device avoiding delivery and guiding catheters.

There is also a need to provide a method for insertion of a self-expandable implantable device. The device is inserted through the skin and the puncturing hole is significantly smaller than 2 mm.

There is also a need to provide a method for insertion of an implantable device under ultrasound guidance.

There is also a need to provide a device for vessel occlusion.

There is also a need to provide a device for opening vessel occlusion and maintaining vessel patency.

There is a need to provide an implantable intravascular drug delivery platform.

There is a need to provide an implantable intravascular radiation delivery platform.

There is a need to provide an implantable embolic protection device.

There is also a need to provide a device for occlusion of a heart orifice, lumen, or atrial appendage.

### SUMMARY OF THE DISCLOSURE

The present disclosure describes embodiments of expandable devices and methods for implanting the devices within the body. More specifically, the present disclosure describes embodiments of devices for at least one of vein occlusion, vein ligation, left atrial appendage and patent foramen ovale occlusion. The disclosure also provides embodiments of devices for at least one of opening a stenotic vessel and maintaining vessel patency. Methods for implanting embodiments of devices according to the present disclosure are also provided.

In some embodiments, the devices are comprised of a mono-filament (hereinafter “cord”) which is spatially bent and/or twisted. The cord is made of a super-elastic metal (e.g., nitinol) and is shaped to a desired three-dimensional configuration as known in the art (e.g., winding around a mandrel, heating, and cooling). In some embodiments, the cord can be coated with at least one of swellable polymers, degradable polymers, drug eluting polymers, radioactive materials, radiolucent and echogenic materials, and other biocompatible materials. The devices according to some embodiments have two operation modes—unexpanded state (un-deployed) and expanded state (deployed). The cord may be implanted using a delivery system comprising a substantially rigid needle having a diameter of about <1 mm (3 French, 0.04″) and preferably includes a sharp distal end. The cord is preassembled within the needle in its unexpanded state, and positioned at the distal end of the sharp end. In some embodiments, the unexpanded state may resemble that of a substantially linear (straight) wire. For example, the unexpanded state may have the shape of a stretched helix. A pusher in the form of an elongated rod may also be preassembled within the needle, extending from the proximal end of the needle to the proximal end of the cord. The implantation of the cord may be performed by piercing the skin and underlying tissues and advancing the needle to the target organ under ultrasound guidance. At the desired location, the cord is exteriorized from the needle by, for example, retraction, pushing, rotating, or twisting of the needle, retraction, pushing, rotating, or twisting the pusher, or any combination thereof, thereby creating relative motion between the needle and the pusher. After retraction of the cord from the needle, the cord, according to some embodiments, assumes the preassembled configuration within target (expanded deployed state).

In some embodiments, the cord resides within a flexible catheter when in the un-deployed contracted state. The catheter is introduced to the target site/organ using a rigid needle and exteriorized at a first location (e.g., renal pelvis) where the catheter is exteriorized and advanced to a second location (ureter). At the second location the cord is exteriorized from the catheter and resumes its final expanded shape (e.g., spiral stent for opening ureter stricture).

Examples for applications in which the cord can be used include any of:
Vessel occlusion
Vessel ligation
Left atrial appendage occlusion
Patent foramen ovale occlusion
Opening or dilating stenosed or strictured vessels, and maintaining vessel patency
Radiotherapy
Imaging marker

### Vessel Occlusion

In some embodiments, the cord can be used for vessel occlusion (e.g., vein, artery, ureter, etc.). In some embodiments, at least one cord is inserted in at least one varicose vein or one or more varicose vein tributaries. In the deployed expanded state, within the vein, the vessel, the cord assumes the shape of a coil, a spring, a skein, a tangle, a bird's nest, or other space-filling spatial configurations. The cord may be made of shape memory alloy and can be covered with swellable polymer (e.g. cladded nitinol core). In some embodiments, one or two ends of the cord pierce the vein walls, thereby providing fixation. The cord ends, located externally to the vein lumen, can be attached to anchors that further secure the cord position and avoid migration. The anchors can be self-expandable and can be made of radiopaque or echogenic material to provide visualization under fluoroscopy or ultrasonography.

In some embodiments, the insertion method includes puncturing the vein at two approximately diametrically-opposed sites on the vein wall, inserting the cord such that it is anchored at both of the opposing puncture sites, whereupon the cord is situated across the vein lumen between the diametrically-opposing puncture sites. The cord can be inserted under ultrasound guidance CT, MRI, or other imaging means known in the art. More than one cord can be implanted across one plane (vein-transverse cross section) or at different locations.

In some embodiments, a method for implanting a spatially bent and/or twisted implant in a patient is presented, with the method comprising providing a mono-filament implant configured to assume an undeployed, substantially linear state and a spatially bent and/or twisted deployed state. The implant includes a proximal and a distal end, and, in some embodiments, the implant substantially corresponds to the lumen of a hollow needle which is used to implant the filament. The method may also include creating a puncture in a vessel of the patient, positioning the distal end of the implant in the vessel through the puncture and converting the implant from the undeployed state to the deployed state such that the proximal end of the implant is proximate the puncture. The implant can be any of: an occlusion device, a delivery platform for a therapeutic agent, a stent, a cavity occlusion device, an embolic protection device, and may comprise at least one anchor.

In some embodiments, a mono-filament implant is provided and is configured to assume an undeployed substantially linear state and a spatially bent and/or twisted deployed state. The implant includes a proximal and a distal end, and may be delivered via a delivery catheter. Such a delivery catheter may comprise a hollow needle having a lumen. In some embodiments, when readying the implant for implantation into a patient, the implant is first provided in its undeployed state within a hollow needle having a lumen, such that, the shape of the implant substantially corresponds to the shape of the needle lumen. In some embodiments, upon positioning of the distal end of the implant in within a vessel via a puncture in the vessel, the implant is deployed and corresponds to the spatially bent and/or twisted deployed state, with the proximal end of the implant being proximate the puncture upon conversion of the implant from the undeployed state to the deployed state.

In some embodiments, a system for realizing at least one of a vessel occlusion, a vessel ligation, a left atrial appendage occlusion, a patent foramen ovale occlusion, and opening or dilating stenosed or strictured vessels and maintaining vessel patency, is provided. Such a system may comprise a mono-filament implant according to any of the disclosed embodiments, a delivery catheter comprising at least a hollow needle of less than about 1 mm in diameter, where the needle is configured to house the mono-filament implant in the undeployed state, and a pusher is configured to push the mono-filament implant from the delivery catheter.

### Vessel Ligation

Vessel ligation is the process of lumen obliteration by adhering vessel walls with one or more suture. In some embodiments, closure of a vein, an artery or any other lumen is performed with the cord. The insertion method may include puncturing a vein wall at two diametrically-opposed sites and retraction the needle away from the cord distal end allowing distal anchor to self-expand. Consequently, the needle may be further retracted exteriorizing the cord within vein lumen and when needle end is retracted to a point external to the vein lumen, the proximal anchor is self-expanded. The final stage of vessel ligation is done by sliding the proximal anchor towards the distal anchor, thus externally compressing and adhering two opposing vein walls. The procedure can be performed at one or more opposing points across the vein lumen.

### Left Atrial Appendage (LAA) Occlusion

The cord can be used as a LAA occluding device (hereinafter “LAA occluder” or “occluder”). In some embodiments, the spatial configuration of the cord in the expanded state is a spiral that is formed by continuous winding with increasing radius of curvature. The spiral can be planar (disc-like), or in a concave or convex configuration. In another embodiment, the occluder can comprise two spiral plates (double disc) that are connected by a connecting neck. The cord can be covered by a swellable polymer that expands after contact with an aqueous environment (e.g., blood). In the expanded state within the body the polymer swells and bridges the gaps between curved wires, thereby creating a sealed plate. Device implantation comprises introducing a delivery needle through an intercostal space, lungs, pericardial space, and into the LAA appendage orifice. When the needle end approaches the LAA orifice the cord is exteriorized by needle retraction and/or cord advancement. The spiral shaped disc (occluder) is deployed at the LAA orifice, thus preventing left atrial blood from entering the LAA. It also prevents internal LAA thrombi from migrating to the left atrium and the systemic circulation. In the double disc configuration the first disc is deployed at the LAA orifice and the second disc is deployed within the LAA appendage and serves as an anchor to secure the first disc in place.

### Patent Foramen Ovale (PFO) Occlusion

The cord can be used as a PFO occluding device (hereinafter “PFO occluder” or “occluder”). In some embodiments, the cord's spatial configuration in the expanded state is the double disc configuration mentioned above (LAA occluder). A short spiral, spring shaped neck provides connection between the discs and applies constant force to maintain discs proximity. In some embodiments, the cord is covered with a swellable polymer. The insertion includes introduction of a delivery needle through an intercostal space, lungs, pericardial space, and into the right or left heart atria in the vicinity of the PFO. When the needle end approaches one side of the PFO (i.e., left atrium), the cord is exteriorized and forms a spatial spiral disc configuration. The needle is further retracted across the PFO orifice and positioned at the other side of the PFO within the lumen of the adjacent atrium (i.e., right atrium). Consequently, the cord is further exteriorized and forms a second spiral disc opposing the first one. Finally the spring neck forces adherence of two discs and occlusion of both sides of the PFO.

### Opening a Vessel and Maintaining Patency

The cord in the expanded state can have a tubular spring shape forming a stent like device or any 3D scaffold (ball shape, conical shape), which apposes the walls of a hollow body cavity and provides force for maintaining lumen (cavity) patency. The insertion includes introduction of a needle into the desired lumen (e.g., stenotic artery, stricture in ureter or GI tract, etc.) and exteriorizing the cord within the lumen.

### Radiotherapy/Drug Therapy

The cord can be coated with any radioactive material known in the art as radiation therapeutic material and deployed within or in the vicinity of a tumor. The “radioactive cord” is introduced through a needle as described above, deployed at desire location and assumes a bird's nest configuration or any other 3D space occupying shape. Similarly, the cord can be coated with any known drug to provide a drug elution platform.

### Imaging Marker

The cord can be used as a marker to improve accuracy of imaging targeted treatments such as radiotherapy for cancer, stereotactic procedures, and their likes. In some embodiments, the cord can comprise a radiopaque material for X-ray guided procedures (CT, fluoroscopy) or an echogenic material for ultrasound guided procedures.

### Advantages of the Invention Over the Prior Art

The present invention has several important advantages over prior art devices, which make it generally safer, less invasive, less expensive, and more convenient for both patients and physicians. Some notable advantages of the present invention over the prior art are detailed below:

Various embodiments of devices according to the present invention can be implanted via a very small puncture (about 0.3-0.8 mm in diameter), whereas even the most advanced prior art devices are implanted via a significantly larger puncture (at least 2.5-3 mm in diameter). As a result the frequency and severity of puncture site complications, such as bleeding, is likely reduced by embodiments of the present disclosure as compared to the prior art.

Embodiments of the present disclosure can be implanted using ultrasound imaging alone (or no imaging at all), whereas prior art endovascularly-implanted devices require fluoroscopic imaging. As a result, the present invention entails no exposure to x-ray and obviates the need for injecting potentially-dangerous x-ray contrast agents. In addition, the implantation procedure time is short and can be done bedside at an outpatient setting, thereby substantially reducing cost and hospital admissions.

Various embodiments of devices according to the present disclosure (LAA and PFO occluders) can be implanted without using prior art intra-cardiac manipulations (crossing the atria septum, etc.), which can be complicated and risky.

### BRIEF DESCRIPTION OF THE DRAWINGS

The invention may be better understood with reference to the accompanying drawings and subsequently provided detailed description:

FIG. 1A depicts the undeployed state of a monofilament occlusion device according to some embodiments of the present disclosure.

FIG. 1B depicts the deployed state of a monofilament occlusion device according to some embodiments of the present disclosure.

FIG. 1C depicts the undeployed state of a monofilament occlusion device comprising anchors, according to some embodiments of the present disclosure.

FIG. 1D depicts the deployed state of a monofilament occlusion device comprising anchors, according to some embodiments of the present disclosure.

FIG. 2A depicts a blood vessel prior to implantation of an occlusion device according to some embodiments of the present disclosure.

FIG. 2B is a schematic side view of an occlusion device according to some embodiments of the present disclosure, deployed in a blood vessel.

FIG. 2C is a schematic view of an occlusion device according to some embodiments of the present disclosure, taken in plane AA of FIG. 2B.

FIGS. 3A-3D depict an apparatus and method according to some embodiments of the present disclosure, which are intended for implanting a monofilament occlusion device according to some embodiments of the present disclosure.

FIG. 4A depicts the undeployed state of a monofilament occlusion device comprising a slidable anchor, according to some embodiments of the present disclosure.

FIG. 4B depicts the deployed state of a monofilament occlusion device comprising a slidable anchor, according to some embodiments of the present disclosure.

FIG. 5A depicts a perpendicular cross section of a body vessel.

FIG. 5B depicts a monofilament occlusion device comprising a slidable anchor according to some embodiments of the present disclosure, deployed in a body vessel.

FIGS. 6A-6E depict an apparatus and method according to some embodiments of the present disclosure, which are intended for implanting a monofilament occlusion device comprising a slidable anchor, according to some embodiments of the present disclosure.

FIG. 7A depicts the undeployed state of a monofilament therapeutic agent delivery platform according to some embodiments of the present disclosure.

FIG. 7B depicts the deployed state of a monofilament therapeutic agent delivery platform according to some embodiments of the present disclosure.

FIG. 8 shows a monofilament therapeutic agent delivery platform according to some embodiments of the present disclosure in operation.

FIG. 9A depicts the undeployed state of a monofilament stent according to some embodiments of the present disclosure.

FIG. 9B depicts the deployed state of a monofilament stent according to some embodiments of the present disclosure.

FIGS. 10A-10D depict an apparatus and method according to some embodiments of the present disclosure, which are intended for implanting a monofilament stent according to some embodiments of the present disclosure.

FIG. 11A depicts the undeployed state of a monofilament cavity occlusion device according to some embodiments of the present disclosure.

FIG. 11B depicts the deployed state of a monofilament cavity occlusion device according to some embodiments of the present disclosure.

FIG. 12A depicts a body cavity prior to the implantation of a cavity occlusion device according to some embodiments of the present disclosure.

FIG. 12B shows a side view of a cavity occlusion device according to some embodiments of the present disclosure, implanted in a body cavity.

FIGS. 13A-13B depict an apparatus and method according to some embodiments of the present disclosure, which are intended for implanting a monofilament cavity occlusion device according to some embodiments of the present disclosure.

### DETAILED DESCRIPTION OF SOME OF THE EMBODIMENTS

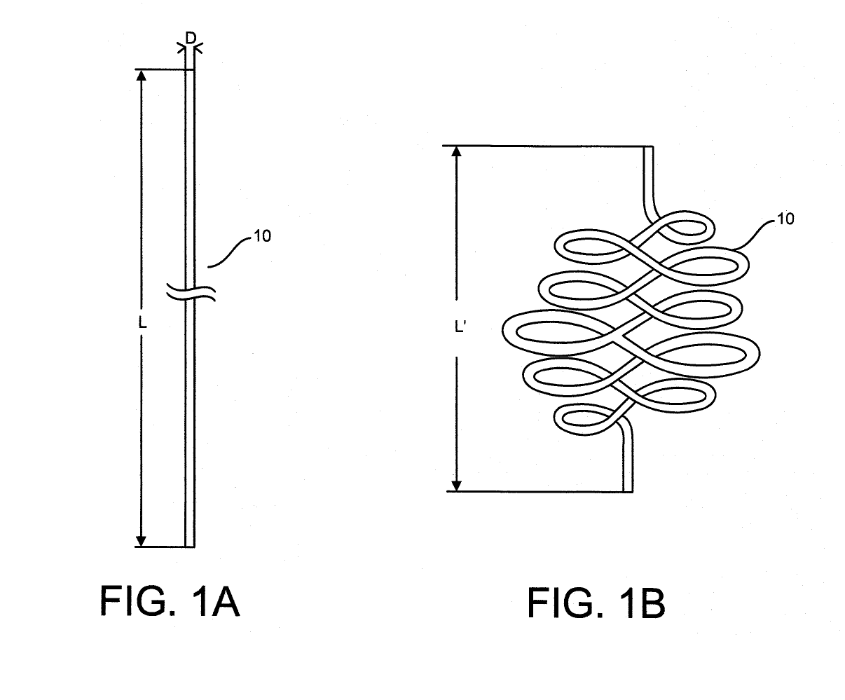

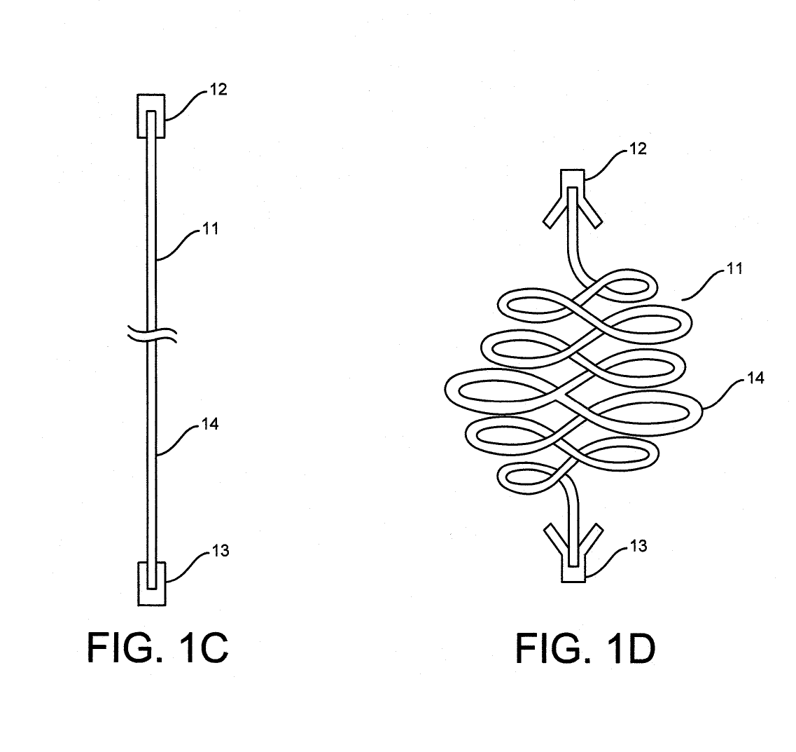

Reference is now made to FIG. 1A, which depicts some embodiments of the undeployed state of a vessel occlusion device of the present disclosure. Occlusion device 10, configured to be implanted in a body vessel, may be a filament of cylindrical shape. However, cross sectional shapes other than circular are also possible.

In some embodiments, the undeployed length L of occlusion device 10 may be greater than the diameter of the body vessel for which it is intended. Thus, if implanting the occlusion device in, for example, a vein or an artery having a diameter of 7 mm, then the length L may be, for example, in the range of about 7 to about 70 mm.

In some embodiments, the diameter D of occlusion device 10 may be substantially less than its length L. For implantation into a blood vessel, the diameter D of the occlusion device may be chosen of a size to fit in the lumen of a thin, hollow needle (for example, a needle whose inner diameter is less than about 1.0 mm). Therefore, the diameter D, according to some embodiments, is less than about 1.0 mm, and more specifically less than about 0.5 mm, and even more specifically, less than about 0.2 mm.

Reference is now made to FIG. 1B, which depicts some embodiments of the deployed state of an occlusion device according to the present disclosure. In the deployed state, occlusion device 10 may assume the shape of a coil, a spring, a skein, a tangle, a bird's nest, or their likes. The deployed length L′ of occlusion device 10 may be greater than the diameter of the body vessel for which it is intended. Thus, if implanting the occlusion device in a vein or an artery having a diameter of 7 mm, then the deployed length L′ may be, for example, in the range of about 7 to about 15 mm.

Occlusion device 10 may be configured to be relatively stiff or, in some embodiments, relatively flexible. Alternatively, occlusion device 10 may be configured to assume any degree of flexibility.

Occlusion device 10, according to some embodiments, may be configured as a solid filament. Alternatively, it may be configured as a tube having a hollow lumen, or as a tube having its ends closed-off, thereby leaving an elongated air-space inside occlusion device 10. Leaving an air-space inside occlusion device 10 may have the advantage of making occlusion device 10 more echogenic and therefore more highly visible by ultrasound imaging. Occlusion device 10 may possess an echogenic marker or a radiopaque marker.

Occlusion device 10 may be made out of any suitable biocompatible material, such as metal, plastic, or natural polymer. Suitable metals include (for example): steel, stainless steel (e.g., 305, 316 L), cobalt chromium alloys (Elgiloy), shape memory alloys (e.g., nitinol), titanium alloys, tantalum, shape memory polymers, or any combination thereof. Suitable plastics include (for example) silicones, polyethylene, polytetrafluoroethylene, polyvinyl chloride, polyurethane, polycarbonate, and any combination thereof. Suitable natural polymers may include collagen, elastin, silk and combinations thereof.

In some embodiments, occlusion device 10 may comprise an absorbable, biodegradable, or bioresorbable material, such as a bioresorbable polymer or a bioresorbable metal. Suitable bioresorbable polymers include polyL-lactide, polyD,L-lactide, polyglycolide, poly ε-caprolactone, 50/50 D,L lactide/glycolide, 82/18 L-lactide/glycolide, 70/30 L-lactide/ε-caprolactone, 85/15 L-lactide/glycolide, 10/90 L-lactide/glycolide, 80/20 L-lactide/D,L-lactide, or any combination thereof. Suitable bioresorbable metals may include magnesium alloy.

Reference is now made to FIGS. 1C and 1D, which respectively represent the undeployed and the deployed states of another embodiment of an occlusion device according to the present disclosure. Occlusion device 11 is substantially similar to occlusion device 10 of FIGS. 1A and 1B, with the following exception: the ends of occlusion device 11 comprise anchors 12 and 13. Each anchor 12 and 13 has an undeployed state, as in FIG. 1C, and a deployed state, as in FIG. 1D. In the undeployed state, occlusion device 11, including anchors 12 and 13, is configured to reside in the lumen of a hollow needle. Upon exteriorization from such a needle, occlusion device 11 may assume the shape of a coil, a spring, a skein, a tangle, a bird's nest, or their likes, and anchors 12 and 13 assume a shape configured to strongly attach to tissue in the vicinity of the occlusion device's implantation site. As a result, migration of occlusion device 11 following its implantation is minimized or even prevented. Ends 12 and 13 may be an integral part of occlusion device 11, or alternatively, they may be components of occlusion device 11. In the latter case, ends 12 and 13 will be connected to filament 14 using for example, an adhesive, a mechanical connection, or any other suitable connection means known in the art. Anchors 12 and 13, which are configured to change their shape as device 11 transitions from the undeployed to the deployed state, may be made of a suitable biocompatible material, such as a metal, a plastic, or a natural polymer. Anchors 12 and 13 may be made of a shape memory alloy or a shape memory polymer.

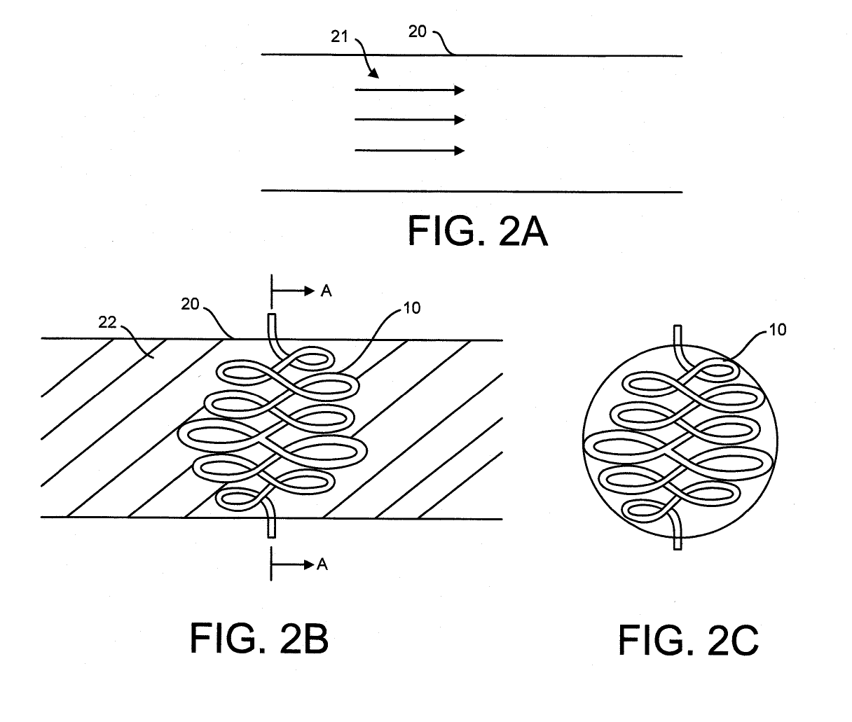

Reference is now made to FIG. 2A, which depicts a schematic side-view of a blood vessel before implantation of occlusion device 10. Reference is also made to FIGS. 2B and 2C, which respectively depict a schematic side view of the blood vessel after implantation of device 10, and a schematic cross-sectional view taken in the plane AA of FIG. 2A.

FIG. 2A shows a patent blood vessel 20, such as an artery or a vein, in which blood 21 is free to flow. Suitable veins may be, for example, perforators of the great saphenous vein. Upon implantation of occlusion device 10 in blood vessel 20, resistance to blood flow 21 is created in blood vessel 20. Whenever occlusion device 10 takes the form of a dense coil, spring, skein, tangle, bird's nest or their likes, and whenever the porosity of deployed occlusion device 10 is sufficiently low, the resistance becomes sufficiently large as to cause blood 21 to stagnate and coagulate. As a result, blood clot or thrombus 22 is formed in the vicinity of occlusion device 10, which completely occludes vessel 20. Blood flow in vessel 20 is thus completely prevented.

It is important to note that occlusion device 10 should sufficiently “fill” the entire cross-section of vessel 20 (FIG. 2C): for, if large gaps remain between device 10 and, for example, the walls of vessel 20, resistance to blood flow will not be sufficient to cause blood 21 to stagnate and coagulate.

Occlusion device 11 works in a substantially similar manner to occlusion device 10, except that anchors 12 and 13 of occlusion device 11 further protect the device against migration.

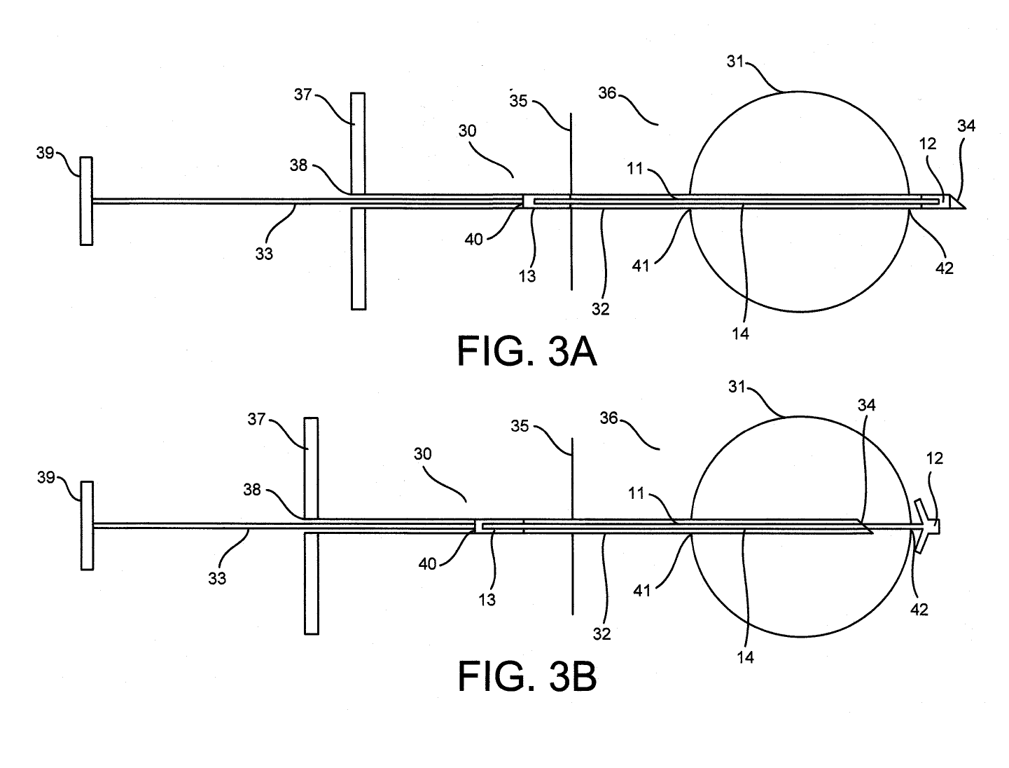

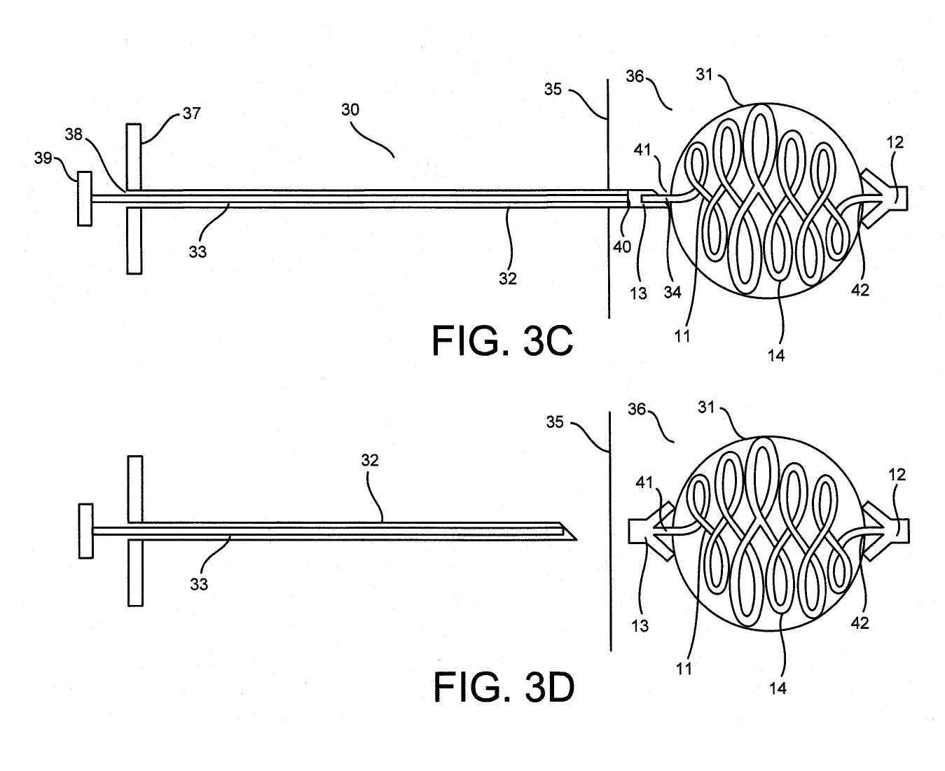

Reference is now made to FIGS. 3A-3E, which describe an apparatus and a method according to some embodiments of the present disclosure for implanting an occlusion device according to some embodiments of the present disclosure. FIG. 3A depicts a delivery device 30 configured to implant occlusion device 11 in body vessel 31. Delivery device 30 comprises a hollow needle 32, a pusher 33, and occlusion device 11. Hollow needle 32 has a sharp end 34 configured to pierce skin 35, subcutaneous tissue 36, and body vessel 31 of a patient. Needle 32 may have a needle handle 37 located at its proximal end 38. The needle handle 37 may be rigidly connected to needle 32. Pusher 33 may have a pusher handle 39 located at its proximal end.

Hollow needle 32 may have a very small inner and outer diameter. For example, if the maximal collapsed diameter of undeployed occlusion device 11 is 200 microns, the inner diameter of hollow needle 32 may be in the range of 200-600 microns, and the outer diameter of hollow needle 32 may be in the range of 300-800 microns. Thus, the puncture holes made by hollow needle 32 in a patient's tissue may be sufficiently small (300-800 microns) as to be self-sealing.

Hollow needle 32 may be made out of any suitable biocompatible material, such as, for example, steel. Pusher 33 may also be made out of a metal such as steel. Handles 37 and 39 may be made out of plastic.

Both occlusion device 11 and pusher 33 are slidable within the lumen of hollow needle 32. Prior to deployment, occlusion device 11 is located inside the lumen of needle 32 near its distal end 34. The distal end 40 of pusher 33 is also located inside the lumen of hollow needle 32. The distal end 40 of pusher 33 is in contact with the proximal end of proximal anchor 13 of occlusion device 11. After deployment, as depicted in FIG. 3D, occlusion device 11 is exteriorized from hollow needle 32, and the distal end 40 of pusher 33 roughly coincides with distal end 34 of hollow needle 32.

The implantation of occlusion device 11 in body vessel 31 may proceed as follows: First, an operator determines that it is desirable to implant occlusion device 11 in body vessel 31. Under the guidance of a suitable imaging modality (not shown), such as, for example, ultrasound, high resolution ultrasound, or CT scanning, or without imaging guidance at all, the operator punctures skin 35 adjacent to vessel 31 using the sharp end 34 of needle 32. Note that delivery device 30 is in the configuration depicted in FIG. 3A, that is, with occlusion device 11 housed near the distal end of hollow needle 32, in its undeployed, substantially linear state, substantially straight-wire state. The operator then carefully advances delivery device 30 through the subcutaneous tissue 36, and transversely punctures vessel 31 at approximately diametrically-opposed sites 41 and 42. The first puncture 41 of vessel 31 is made on its side closer to skin 35, and the second puncture 42 is made on the diametrically-opposite side. Note that the second puncture 42 may be either complete or partial: Sharp end 34 of needle 32 may completely traverse the wall of vessel 31, or alternatively, only breach the inside (lumen side), but not the outside of the wall. The sharp end 34 of needle 32 may then be advanced a few more millimeters interiorly into the patient. This situation is depicted in FIG. 3A.

Next, the operator holds pusher 33 substantially motionless while retracting hollow needle 32 backwards, away from the patient. This may be done with one hand: the thumb of the operator pushes on pusher handle 39, whereas one or more fingers grasp needle handle 37. Thus, the distal end 34 of hollow needle 32 is retracted over occlusion device 11. In this way distal anchor 12 of device 11 is exteriorized from needle 32. It then assumes its deployed state in the tissue proximate second puncture 42, thereby anchoring the distal end of device 11 in the tissue. This situation is depicted in FIG. 3B.

It is noted that all absolute and relative motions of needle 32 and pusher 33 may be made using an automated mechanism, such as, for example, an automated electro-mechanical mechanism (not shown).

To exteriorize the remainder of occlusion device 11 from hollow needle 32, the operator serially or simultaneously causes pusher 33 to be pushed and/or needle 32 to be retracted. As device 11 is thus exteriorized from the needle, it assumes its deployed shape. According to some such embodiments, the tip of needle 32 is not retracted exteriorly from the lumen of vessel 31. The operator terminates the push-pull motion once filament 14 is essentially exteriorized from needle 32 into the lumen of vessel 31, and anchor 12 is situated, still inside the lumen of needle 32, at its implantation site. The situation is then as depicted in FIG. 3C.

To complete the implantation procedure, the operator once again holds pusher 33 steady while causing needle 32 to be retracted over the pusher. This causes the proximal anchor 13 to be exteriorized at its implantation site and assume its deployed shape. Once the entire device 11 is thus exteriorized and implanted in its deployed state, both needle 32 and pusher 33 are exteriorized from the patient's body. This completes the implantation procedure, as depicted in FIG. 3D. Note that because both the occlusion device 11 and hollow needle 32 are of a diameter which is sufficiently small (< about 1 mm), all of the holes of and the punctures made in body tissues during the procedure may be self-sealing. Therefore, the suturing of holes and punctures thus made is unnecessary. If it is determined that one or more additional occlusion members should be implanted in one or more additional implantation sites, the procedure may be performed again, essentially as described above.

The delivery device and implantation method corresponding to embodiment 10 of the occlusion device are substantially similar to those described for delivery device 30 and its associated method of use, as described above. Therefore, detailed descriptions of a delivery device and an implantation procedure corresponding to occlusion device 10 are omitted.

It is emphasized that in some embodiments, implantable occlusion devices 10 and 11, taken together with their delivery means 30, share the following characteristics: (i) The puncture holes made by delivery device 30 are sub-millimetric, and are therefore self-sealing and self-healing; (ii) The implant (that is, the implantable occlusion devices) assume the form of substantially straight wire (monofilament) when in their undeployed state; (iii) The implant is implanted in the immediate vicinity of a vessel puncture site.

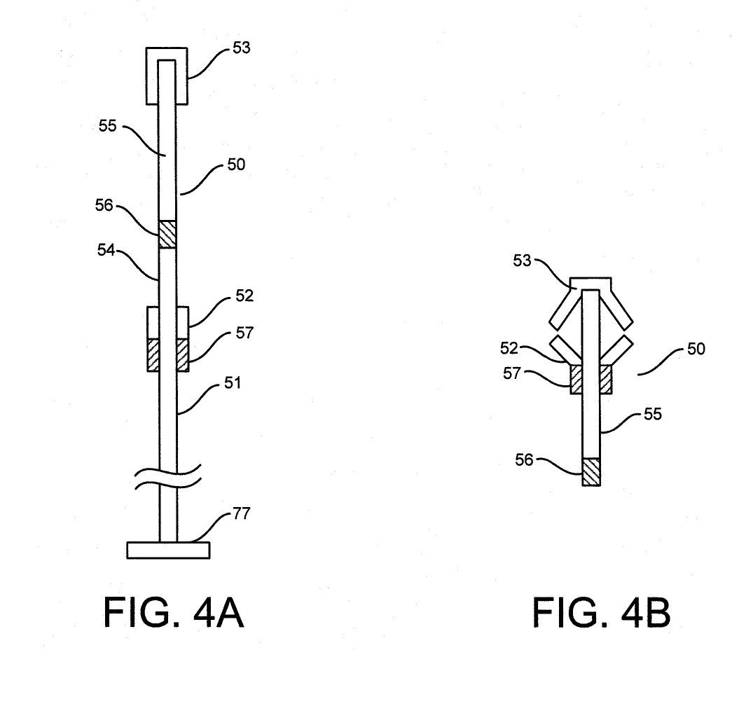

Reference is now made to FIGS. 4A and 4B, which describe the undeployed and the deployed states, respectively, of a body-vessel occlusion device according to some embodiments of the present disclosure.

Occlusion device 50 of FIG. 4A may comprise a filament 51, a proximal anchor 52, and a distal anchor 53. Filament 52 may be separated into a proximal part 54 and a distal part 55 at separation point 56. The proximal and distal parts 54 and 55 are initially connected at separation point 56, and can be disconnected upon the application of external force or signal. A removable handle 77 may optionally be attached to proximal part 54 at its proximal end.

The initial connection between parts 54 and 55 may be mechanical. For example, part 54 may screw into part 55, and disconnection of the parts may be brought about by unscrewing them. Alternatively, filament 55 may comprise a conducting core cladded with an insulating layer at every point along its length except for separation point 56. When it is desired to separate parts 54 and 55, electrical current from an external source (not shown) is run through filament 55, thereby causing electorlysis and subsequent disconnection of parts 54 and 55 at separation point 56.

Proximal anchor 52 may slidable over filament 51. For example, proximal anchor 52 may comprise a slidable element 57 configured to slide over filament 51. Slidable element 57 may comprise a locking mechanism that fixes it in a desired location along filament 51. Suitable locking mechanisms known to those of skill in the art.

In its undeployed state, occlusion device 50 is configured to reside in the lumen of a fine needle, substantially collinear with the lumen of the needle. The anchors 53 and 57 assume their undeployed configuration in the undeployed state.

The undeployed length of occlusion device 50 may be in the range of several centimeters to 100 cm. The diameter of occlusion device 50 is preferably less than 1.0 mm. In particular, the diameter of occlusion device 50 is less than 0.5 mm, and even more particularly, less than 0.2 mm. Separation point 56 is typically located between 1 mm and 30 mm from the distal end of occlusion device 50.

In the deployed state of occlusion device 50 (FIG. 4B), anchors 52 and 53 are in their deployed configuration. Anchor 52 is moved towards anchor 53 such that the distance between them is typically between 1 mm and 10 mm. The most proximal point of anchor 52 is distal to separation point 56. Proximal part 54 of filament 51 is separated from distal part 55. Thus, the deployed state of occlusion device 50 comprises distal part 55 of filament 51 and no longer comprises the proximal part 54.

Occlusion device 50 may be configured to be relatively stiff or, in some embodiments, relatively flexible. Alternatively, occlusion device 50 may be configured to assume any degree of flexibility. Stiffness and diameter along the length of filament 50 may be variable.

Occlusion device 50, according to some embodiments of the present disclosure, may be configured as a solid filament. Alternatively, it may be configured as a tube having a hollow lumen, or as a tube having its ends closed-off, thereby leaving an elongated air-space inside occlusion device 50. Leaving an air-space inside occlusion device 50 may have the advantage of making occlusion device 50 more echogenic and therefore more highly visible by ultrasound imaging. Occlusion device 50 may possess an echogenic marker or a radiopaque marker.

Occlusion device 50 may be made out of any suitable biocompatible material, such as metal, plastic, or natural polymer. Suitable metals include (for example): steel, stainless steel (e.g., 305, 316 L), cobalt chromium alloys (Elgiloy), shape memory alloys (e.g., nitinol), titanium alloys, tantalum, shape memory polymers, or any combination thereof. Suitable plastics include (for example) silicones, polyethylene, polytetrafluoroethylene, polyvinyl chloride, polyurethane, polycarbonate, and any combination thereof. Suitable natural polymers may include collagen, elastin, silk and combinations thereof.

In some embodiments, occlusion device 50 may comprise an absorbable, biodegradable, or bioresorbable material, such as a bioresorbable polymer or a bioresorbable metal. Suitable bioresorbable polymers include polyL-lactide, polyD,L-lactide, polyglycolide, poly ε-caprolactone, 50/50 D,L lactide/glycolide, 82/18 L-lactide/glycolide, 70/30 L-lactide/ε-caprolactone, 85/15 L-lactide/glycolide, 10/90 L-lactide/glycolide, 80/20 L-lactide/D,L-lactide, or any combination thereof. Suitable bioresorbable metals may include magnesium alloy.

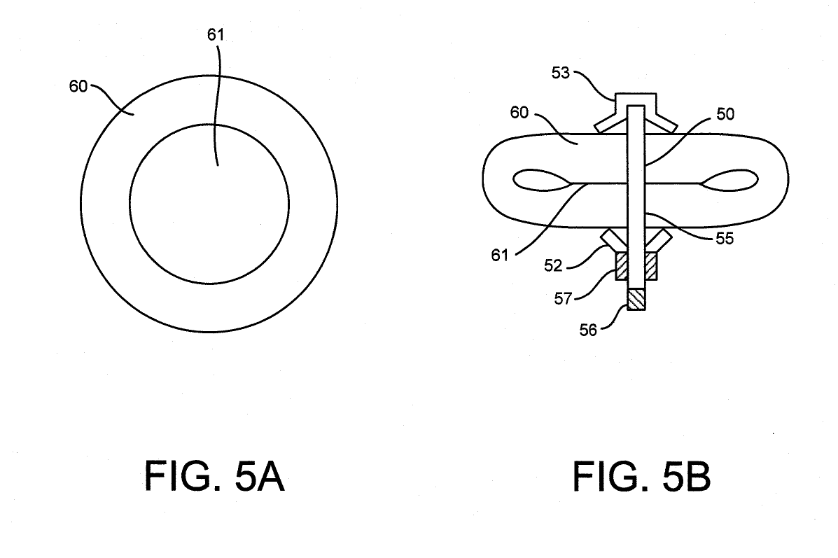

Reference is now made to FIG. 5A, which depicts a schematic cross-sectional view of a blood vessel before implantation of occlusion device 50. Reference is also made to FIG. 5B, which depicts a schematic cross-sectional view of the blood vessel after implantation of device 50.

FIG. 5A shows the circular cross-section of a patent blood vessel 60, such as an artery or a vein, in which blood is free to flow in vessel lumen 61. Suitable veins may be, for example, perforators of the great saphenous vein. Upon implantation of occlusion device 50 in blood vessel 60 (FIG. 5B), anchors 53 and 57, which are brought close together, push against opposite sides of the vessel wall, thereby flattening a cross section of the vessel. As a result, lumen 61 disappears, or substantially disappears. Thus, occlusion device 50 causes vessel 60 to become either totally or substantially occluded.

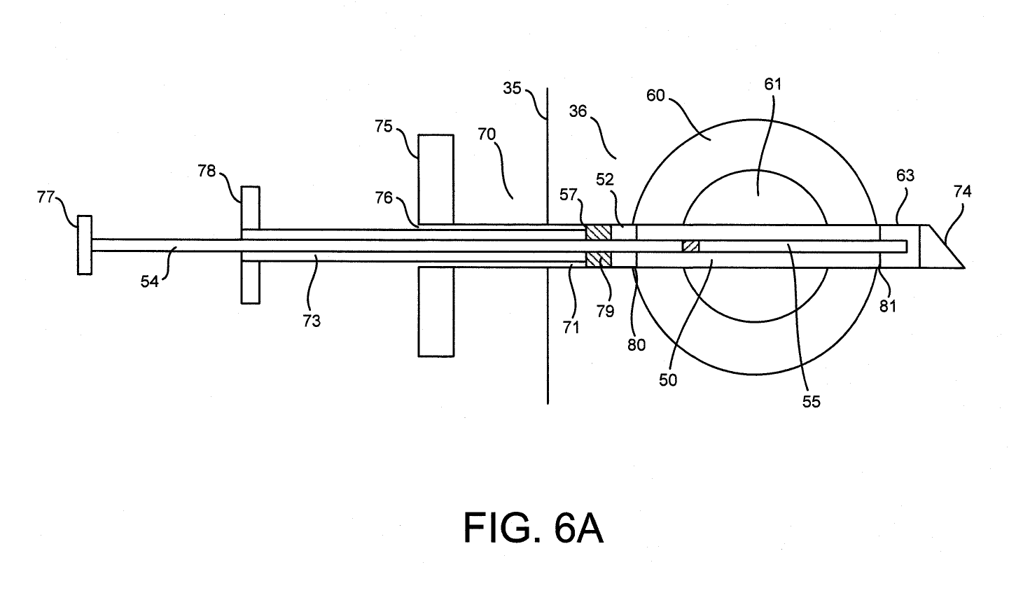

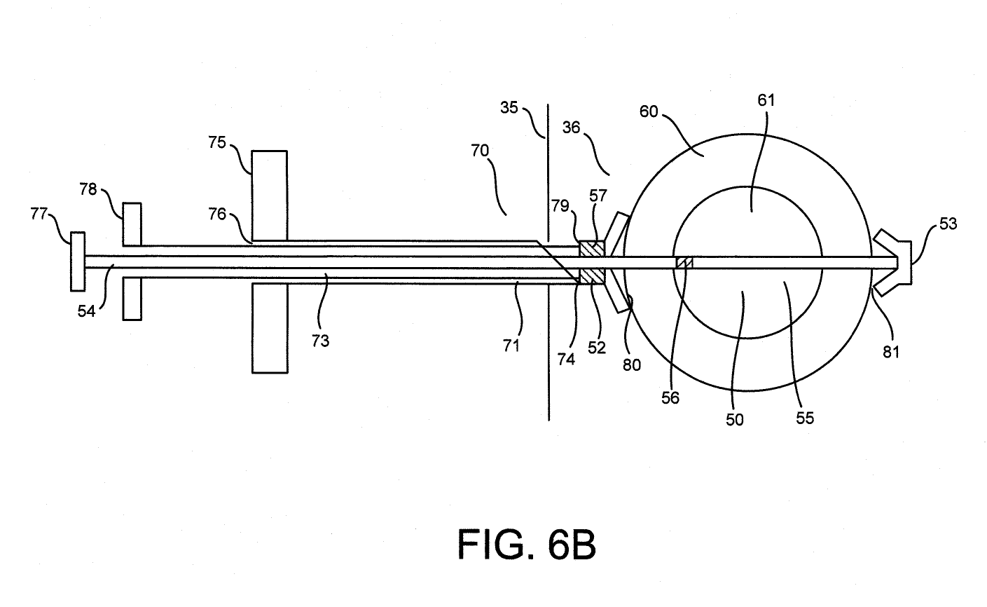

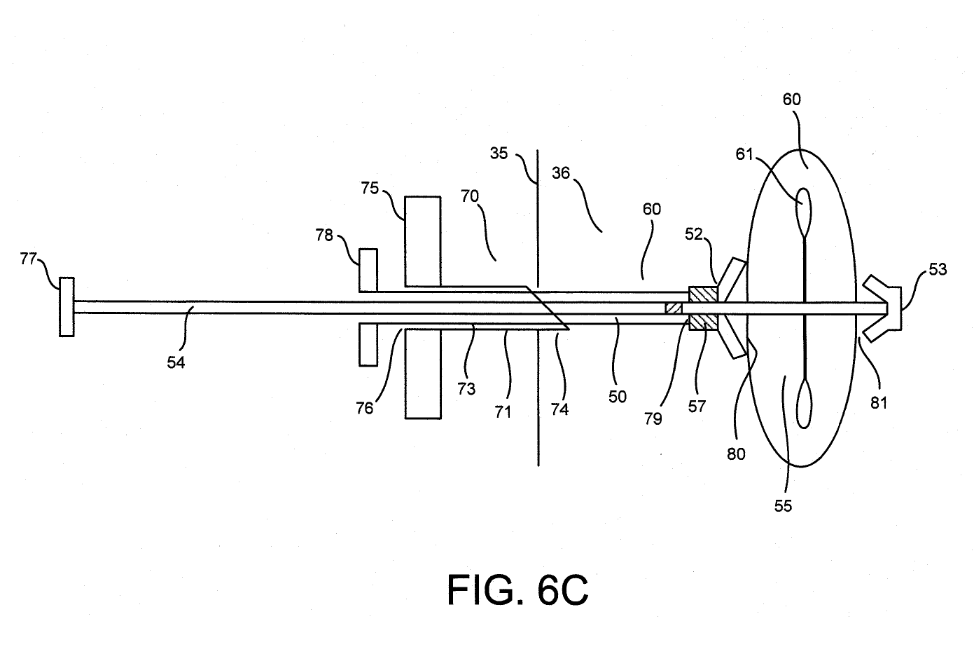

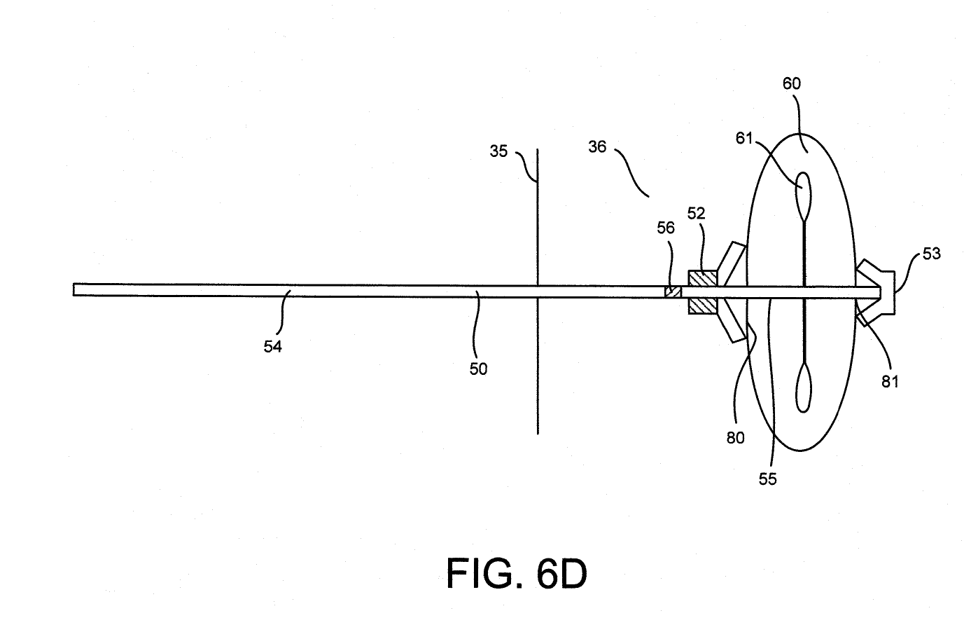

Reference is now made to FIGS. 6A-6E, which describe an apparatus and a method according to some embodiments of the present disclosure for implanting an occlusion device according to some embodiments of the present disclosure. FIG. 6A depicts a delivery device 70 configured to implant occlusion device 50 in body vessel 60. Delivery device 70 comprises a hollow needle 71, push tube 73, and occlusion device 50. Hollow needle 71 has a sharp end 74 configured to pierce skin 35, subcutaneous tissue 36, and body vessel 60 of a patient. Needle 71 may have a needle handle 75 located at its proximal end 76. The needle handle 75 may be rigidly connected to needle 71. Push tube 73 may have a push tube handle 78. The push tube handle 78 may be rigidly connected to push tube 73.

Hollow needle 71 may have a very small inner and outer diameter. For example, if the maximal collapsed diameter of undeployed occlusion device 11 is 200 microns, the inner diameter of hollow needle 71 may be in the range of 200-600 microns, and the outer diameter of hollow needle 71 may be in the range of 300-800 microns. Thus, the puncture holes made by hollow needle 71 in a patient's tissue may be sufficiently small (300-800 microns) as to be self-sealing.

Hollow needle 71 may be made out of any suitable biocompatible material, such as, for example, steel. Push tube 73 may also be made out of a metal such as steel. Handles 75, 77, and 78 may be made out of plastic.

Occlusion device 50 and push tube 73 are both slidable within the lumen of hollow needle 71. Occlusion device 50 is also slidable within the lumen of push tube 73.

Prior to deployment, occlusion device 50 is slidably received inside the lumen of push tube 73. The distal end 79 of push tube 73 is in contact with the proximal end of slidable element 57 of anchor 52. Both occlusion device 50 and push tube 73 are slidably received in the lumen of needle 71. The distal anchor 53 of occlusion device 50 is located near the sharp end 74 of needle 71.

The implantation of occlusion device 50 in body vessel 60 may proceed as follows: First, an operator determines that it is desirable to implant occlusion device 50 in body vessel 60. Under the guidance of a suitable imaging modality (not shown), such as, for example, ultrasound, high resolution ultrasound, or CT scanning, or without imaging guidance at all, the operator punctures skin 35 adjacent to vessel 60 using the sharp end 74 of needle 71. Note that delivery device 70 is in the configuration depicted in FIG. 6A, that is, with the distal end of occlusion device 50 near the distal end of hollow needle 71, and in its undeployed, substantially-linear, substantially-straight wire state. The operator then carefully advances delivery device 70 through the subcutaneous tissue 36, and transversely punctures vessel 60 at approximately diametrically-opposed sites 80 and 81. The first puncture 80 of vessel 60 is made on its side closer to skin 35, and the second puncture 81 is made on the diametrically-opposite side. Note that the second puncture 81 may be either complete or partial: Sharp end 74 of needle 71 may completely traverse the wall of vessel 60, or alternatively, only breach the inside (lumen side), but not the outside of the wall. The sharp end 74 of needle 71 may then be advanced a few more millimeters interiorly into the patient. This situation is depicted in FIG. 6A.

Next, by means of handles 75, 77 and 78, the operator holds occlusion device 50 and push tube 73 substantially motionless while retracting hollow needle 71 backwards, away from the patient. Thus, the distal end 74 of hollow needle 71 is retracted over occlusion device 50 and push tube 73 until both anchors 52 and 53 are exteriorized from needle 71. Anchor 53 may then be exteriorized distally to the lumen 61, and anchor 52 may be exteriorized proximally to the lumen 61. Each anchor assumes its deployed state following exteriorization. This situation is depicted in FIG. 6B.

It is noted that all absolute and relative motions of device 50, needle 71 and push tube 73, may be made using an automated mechanism, such as, for example, an automated electro-mechanical mechanism (not shown).

In the next step, by means of handles 75, 77, and 78, the operator holds occlusion device 50 and needle 71 substantially motionless while advancing push tube 73 towards distal anchor 53. Push tube 73 thus pushes proximal anchor 52, causing it to slide towards distal anchor 53. The operator continues to advance push tube 73 until proximal anchor 53 slides past separation point 56 and the distance between anchors 52 and 53 is sufficiently small as to flatten vessel 60 and annul its lumen 61, either totally or partially, as desired. Slidable anchor 52 is then locked in place and cannot slide proximally. This situation is depicted in FIG. 6C.

Next, the operator removes removable handle 77 from proximal part 54 of occlusion device 50. The operator then exteriorizes from the patient's body both needle 71 and push tube 73 over both distal part 55 and proximal part 54 of device 50. The situation is depicted in FIG. 6D.

In the next step, the operator disconnects proximal part 54 of device 50 from the remainder of the device. Disconnection may be brought about by, for example, unscrewing part 54 from part 55. If, for example, filament 54 of device 50 has an electricity-conducting core and an insulating cladding everywhere except separation point 56, the operator may separate parts 54 and 55 by running a sufficiently high electric current in the filament. Finally, the operator exteriorizes part 54 from the patient's body, which completes the implantation procedure (FIG. 6E).

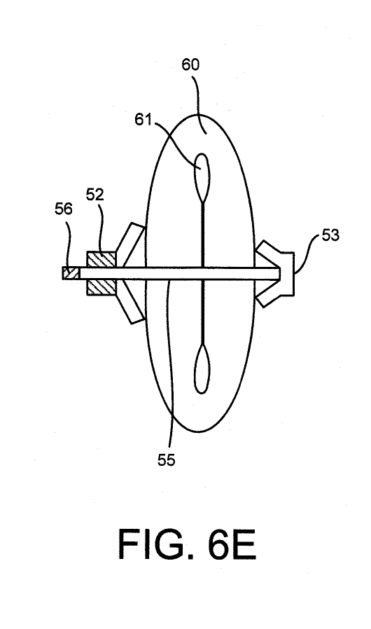

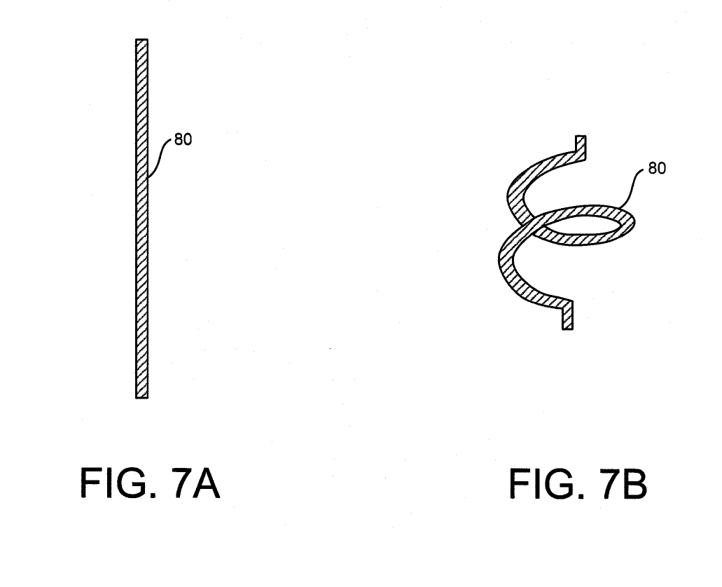

Reference is now made to FIGS. 7A and 7B, which depict the undeployed and the deployed states, respectively, of an implantable therapeutic agent delivery platform according to the present invention.

Therapeutic agent delivery platform 80 is configured to be implanted in a body vessel, such as, for example, a blood vessel, a vein, an artery, a urinary tract vessel, a renal pelvis, or a biliary tract vessel. Therapeutic agent delivery platform 80 can be a shaped as a filament of cylindrical shape. However, cross sectional shapes other than circular are also possible.

The geometry of delivery platform 80 may be substantially similar to the geometry of occlusion device 10, with the following exception: The geometry of delivery platform 80 is configured to allow the free and safe passage of body fluids through the vessel in which it is implanted. For example, if the vessel is a blood vessel, then the geometry of delivery platform 80 will allow the safe passage of blood through the blood vessel, without unwarranted thrombotic events. For example, delivery platform may be shaped as a spring or a coil in which the pitch (vertical distance between consecutive windings) is much greater than the wire thickness. Suitable pitch and wire thickness may be in the range of 1-10 mm and 0.05-0.5 mm, respectively. Wire length may be, for example, 10-100 mm in the undeployed state.

Delivery platform 80, according to some embodiments of the present disclosure, may be configured as a solid filament, a tube having a hollow lumen, or as a tube having its ends closed-off. Delivery platform 80 may possess an echogenic marker or a radiopaque marker. Delivery platform 80 may comprise any of the materials that occlusion device 10 may comprise.

Delivery platform 80 may comprise a therapeutic agent such as a drug or a radiation source. The therapeutic agent may be loaded into the bulk of delivery platform 80, or it may be loaded onto the surface of delivery platform 80. Alternatively, the therapeutic agent may be loaded into a special coating deposited on delivery platform 80.

Delivery platform 80 may comprise, for example, drugs such as fast release drugs, slow release drugs, chemotherapeutic drugs, antibiotics, anti-inflammatories, anti-coagulants, and immunosuppressants. It may also comprise radioactive substances configured to emit therapeutic radiation such as alpha radiation, beta radiation, gamma radiation, or x-rays. The therapeutic agent may be released from delivery platform 80 according to a predetermined time profile. For example, the dose released as a function of time may be initially high and then decay. Alternatively, the dose released may be initially low, increase to a peak, and then decay. Many other predetermined time release profiles are possible by, for example, manipulating the concentration of the therapeutic agent as a function of depth from the surface of delivery platform 80.

Delivery platform 80 may possess anchors. Such anchors, and their connection to the main body of delivery platform 80, may be substantially similar to those of occlusion device 11.

Delivery platform 80 may be implanted in a body vessel in a manner substantially similar to occlusion devices 10 and 11. Delivery platform 80 may lie in the lumen of its delivery needle in an undeployed state resembling a substantially straight wire (FIG. 7A), and assume its deployed shape upon being exteriorized from the needle (FIG. 7B).

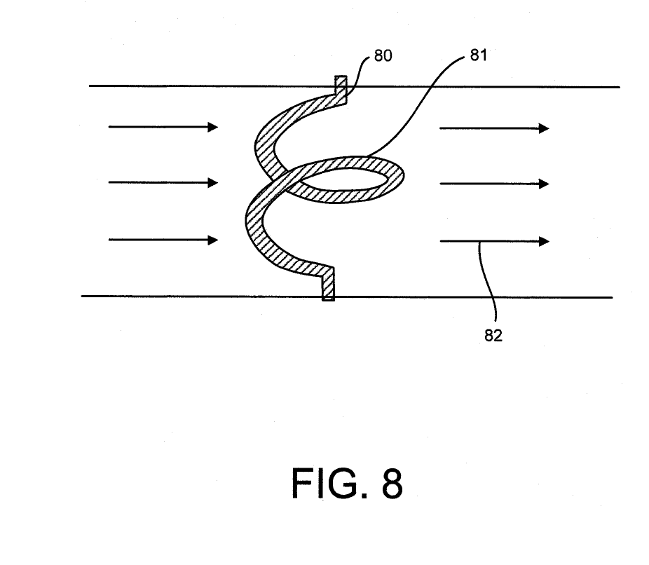

Reference is now made to FIG. 8, which depicts delivery platform 80 implanted inside a body vessel in which a body fluid flows. Delivery platform 80 may release a therapeutic agent 81 such as a drug to body fluid 82 flowing in the vessel. The therapeutic agent 81 may thus be carried to a target organ in selective fashion, thereby limiting unwarranted systemic side effects.

Delivery platform 80 may be particularly suitable for implantation in locations that are difficult or impossible to access in endoluminal fashion, and which are relatively easily accessed by a thin (sub millimetric) implantation (delivery) needle. Suitable implantation locations may include the portal vein (which is virtually inaccessible using endoluminal transcatheter techniques), and the kidney pelvis, which may be accessed using endoluminal transcatheter techniques through the urethra, bladder, and a ureter, but with great difficulty for the operator and at great discomfort for the patient.

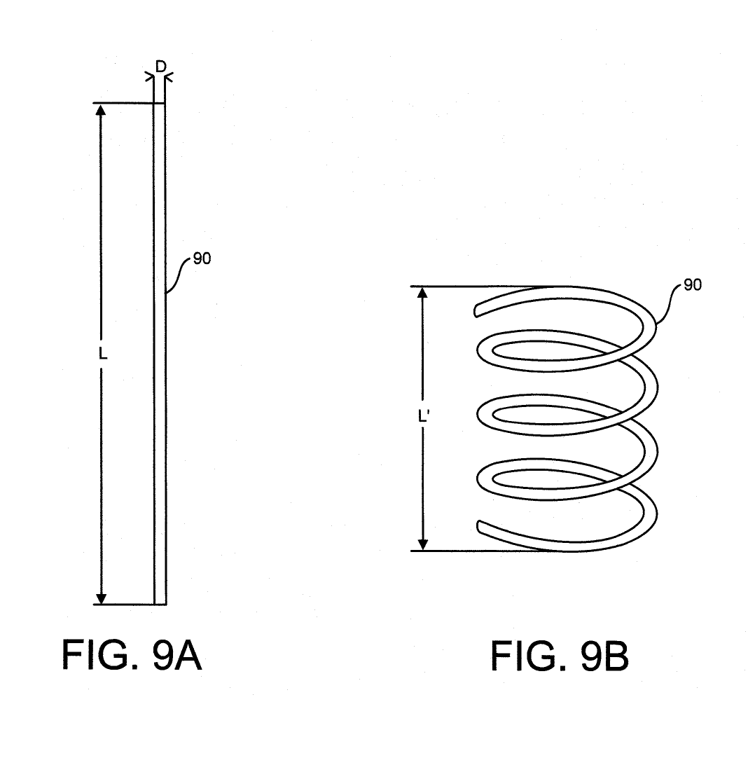

Reference is now made to FIGS. 9A and 9B, which depict the undeployed and the deployed states of a stent 90 according to some embodiments of the present disclosure. In the undeployed state (FIG. 9A), stent 90 may resemble substantially linear or straight wire or filament (or, for example, stretched helix). In the deployed state (FIG. 9B), stent 90 may resemble a cylindrical spring or coil. However, all other shapes that may be constructed by bending and/or twisting a monofilament to occupy a cylindrical shell are also possible.

Stent 90, configured to be implanted in a body vessel and to provide radial support force to its walls, may comprise a filament of cylindrical shape. However, cross sectional shapes other than circular are also possible. In some embodiments, the undeployed length L of stent 90 may be in the range of 2-50 times the perimeter of the body vessel in which it is implanted. For example, if the diameter of the target vessel is 4 mm then the undeployed length L of stent 90 may be in the range of 20-700 mm. The deployed length L′ of stent 90 may be in the range of 2-20 times the diameter of the target vessel. For example, if the diameter of the target vessel is 4 mm then the deployed length L′ may be in the range of 8-160 mm.

In some embodiments, the diameter D of stent 90 may be substantially less than its length L. For implantation into a blood vessel, the diameter D of the occlusion device may be chosen of a size to fit in the lumen of a thin needle (for example, a needle whose inner diameter is less than about 1.0 mm). Therefore, the diameter D, according to some embodiments is less than about 1.0 mm, and more specifically less than about 0.5 mm, and even more specifically, less than about 0.2 mm.

Stent 90, according to some embodiments, may be configured as a solid filament, a tube having a hollow lumen, or as a tube having its ends closed-off. Stent 90 may possess an echogenic marker or a radiopaque marker. Stent 90 may comprise any of the materials that occlusion device 10 may comprise. Stent 90 may be configured to deliver a therapeutic agent, such as a drug or radiation, in substantially the same fashion as delivery platform 80.

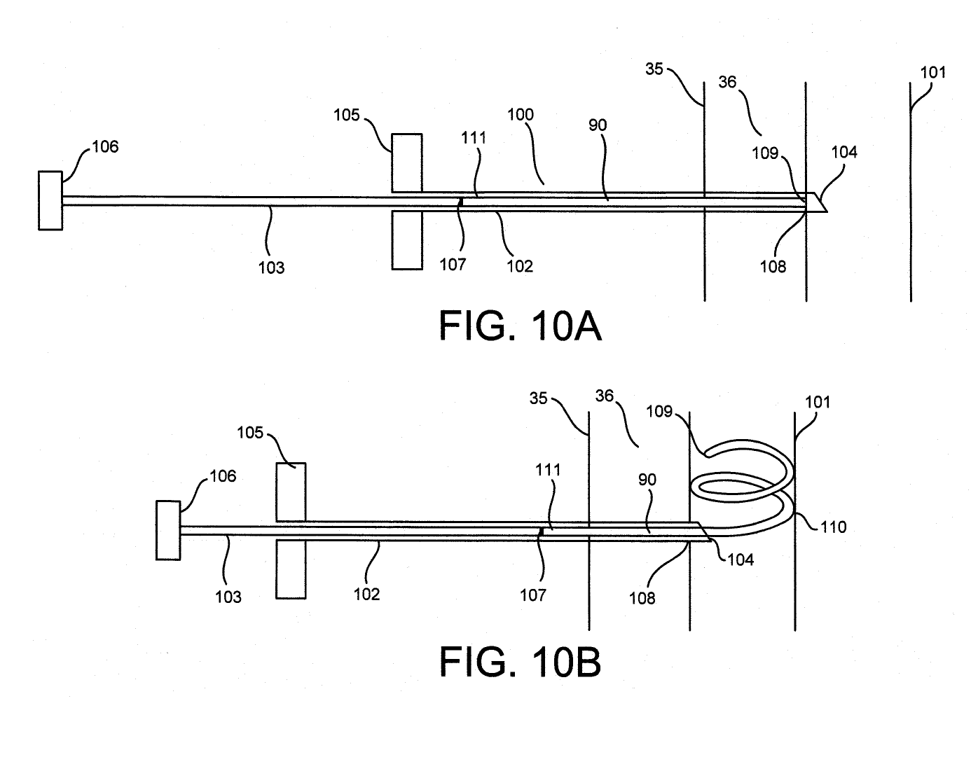

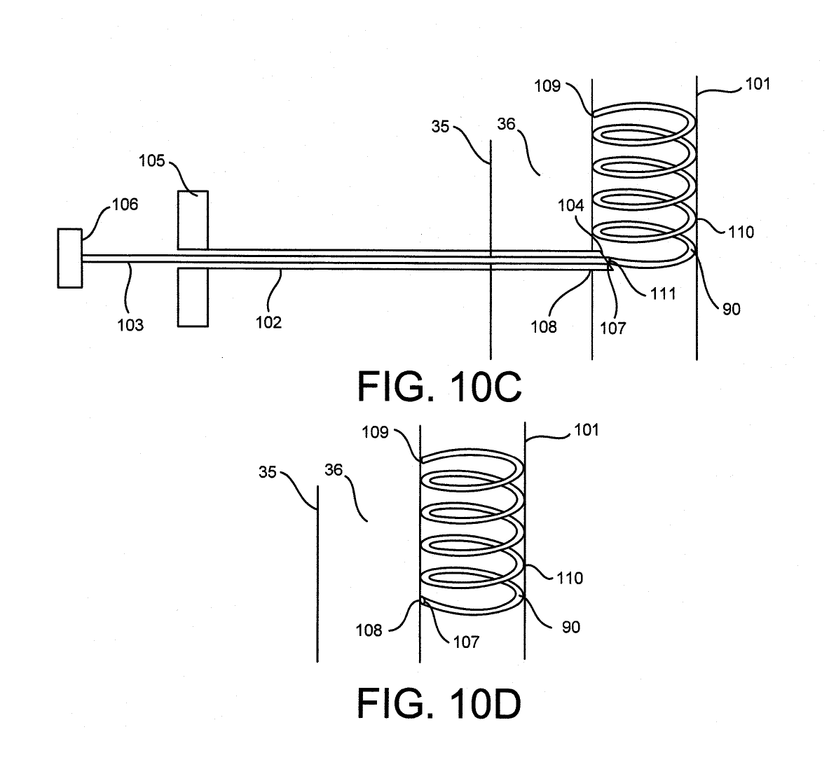

Reference is now made to FIGS. 10A-10D, which describe an apparatus and a method according to some embodiments of the present disclosure for implanting a stent according to some embodiments of the present disclosure. FIG. 10A depicts a delivery device 100 configured to implant stent 90 in body vessel 101. Delivery device 90 comprises a hollow needle 102, a pusher 103, and stent 90. Hollow needle 102 has a sharp end 104 configured to pierce skin 35, subcutaneous tissue 36, and body vessel 101 of a patient. Needle 102 may have a needle handle 105 located at its proximal end. The needle handle 105 may be rigidly connected to needle 102. Pusher 103 may have a pusher handle 106 located at its proximal end.

Hollow needle 102 may have a very small inner and outer diameter. For example, if the maximal collapsed diameter of undeployed stent 90 is 200 microns, the inner diameter of hollow needle 102 may be in the range of 200-600 microns, and the outer diameter of hollow needle 102 may be in the range of 300-800 microns. Thus, the puncture holes made by hollow needle 102 in a patient's tissue may be sufficiently small (300-800 microns) as to be self-sealing and self-healing.

Hollow needle 102 may be made out of any suitable biocompatible material, such as, for example, steel. Pusher 103 may also be made out of a metal such as steel. Handles 105 and 106 may be made out of plastic.

Both stent 90 and pusher 103 are slidable within the lumen of hollow needle 102. Prior to deployment, stent 90 is located inside the lumen of needle 102 near its distal end 104. The distal end 107 of pusher 103 is also located inside the lumen of hollow needle 102. The distal end 107 of pusher 103 is in contact with the proximal end of stent 90. After deployment, as depicted in FIG. 10C, stent 90 is exteriorized from hollow needle 102, and the distal end of pusher 103 roughly coincides with distal end 104 of hollow needle 102.

The implantation of stent 90 in body vessel 101 may proceed as follows: First, an operator determines that it is desirable to implant stent 90 in body vessel 101. Under the guidance of a suitable imaging modality (not shown), such as, for example, ultrasound, high resolution ultrasound, or CT scanning, or without imaging guidance at all, the operator punctures skin 35 adjacent to vessel 101 using the sharp end 104 of needle 102. Note that delivery device 100 is in the configuration depicted in FIG. 10A, that is, with stent 90 housed near the distal end of hollow needle 102, in its undeployed, linear, substantially-straight wire state. The operator then carefully advances delivery device 100 through the subcutaneous tissue 36, and transversely punctures vessel 101 at proximal puncture site 108. The tip 104 of needle 102 slightly protrudes into the lumen of vessel 101. This situation is depicted in FIG. 10A.

Next, the operator holds needle 102 substantially motionless while advancing pusher 103 towards the patient. This may be done with one hand: the thumb of the operator pushes on pusher handle 106, whereas one or more fingers grasp needle handle 105. Thus, the distal end 109 of stent 90 is advanced into the lumen of vessel 101. As stent 90 is exteriorized from needle 102, it assumes a cylindrical spring shape and apposes the walls of vessel 101. Generally, stent 90 will touch the wall of vessel 101 at a location 110 close to a point diametrically opposed to puncture site 108. This situation is depicted in FIG. 10B.

It is noted that all absolute and relative motions of pusher 33 may be made using an automated mechanism, such as, for example, an automated electro-mechanical mechanism (not shown).

To exteriorize the remainder of stent 90 from hollow needle 102, the operator continues to advance pusher 103 distally while holding needle 102 in place. As stent 90 is exteriorized from the needle, it assumes its deployed, coil-like shape. Once the distal end of pusher 103 reaches the distal end of needle 102 stent 90 is completely deployed. Its proximal end 111 resides at a point close to puncture site 108. The situation is then as depicted in FIG. 10C.

To complete the procedure, the operator simultaneously retracts hollow needle 102 and pusher 103 from the patient's body (FIG. 10 D).

It is emphasized that, in some embodiments, stent 90, taken together with its delivery means 100, share the following characteristics: (i) The puncture hole made by delivery device 100 is sub-millimetric, and is therefore self-sealing and self-healing; (ii) The implant (that is, the implantable stent) assumes the form of substantially linear, substantially straight wire (monofilament) when in its undeployed state; (iii) The implant is implanted in the immediate vicinity of the vessel puncture site.

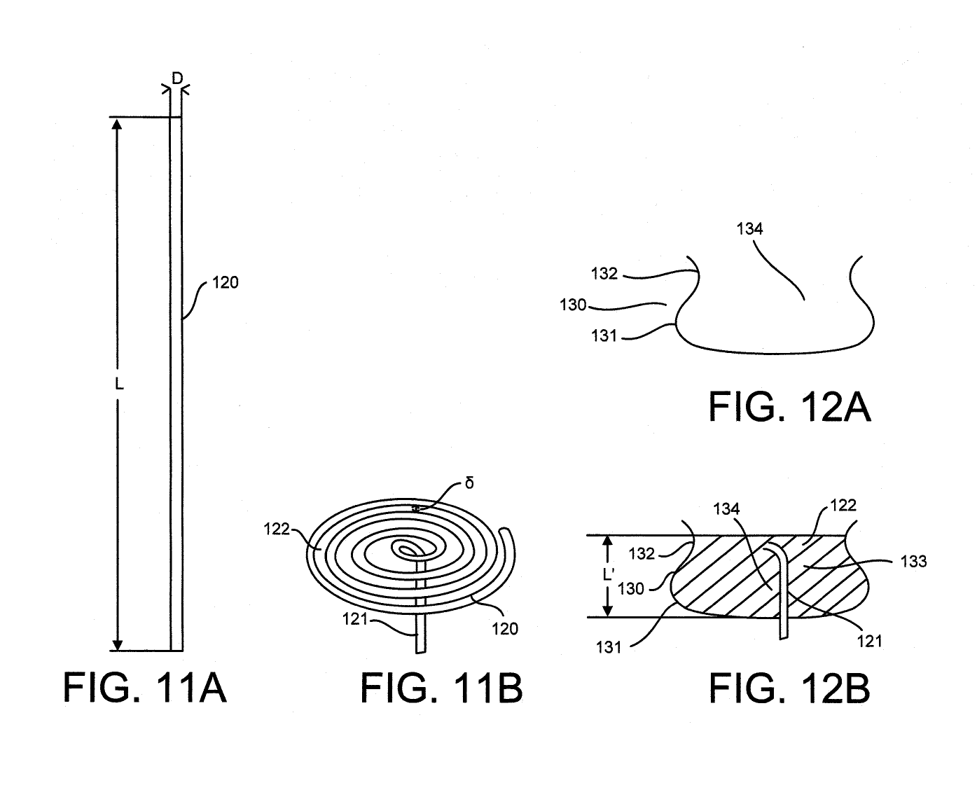

Reference is now made to FIGS. 11A and 11B, which depict the undeployed and the deployed states, respectively, of a body cavity occlusion device 120. Cavity occlusion device 120 is directed at occluding body cavities such as, for examples, a left atrial appendage and an aneurysm. Cavity occlusion device 120 may be a filament of cylindrical shape. However, cross sectional shapes other than circular are also possible.

In some embodiments, the undeployed length of cavity occlusion device 120 may be greater than the size or depth of the body cavity for which it is intended. Thus, if implanting the occlusion device in, for example, a left atrial appendage having a depth L′ of 20 mm, then the length L may be, for example, in the range of about 20 to about 300 mm.

In some embodiments, the diameter D of cavity occlusion device 120 may be substantially less than its length L. The diameter D of the occlusion device may be chosen of a size to fit in the lumen of a thin needle (for example, a needle whose inner diameter is less than about 1.0 mm). Therefore, the diameter D, according to some embodiments is less than about 1.0 mm, and more specifically less than about 0.5 mm, and even more specifically, less than about 0.2 mm.

Reference is now made to FIG. 11B, which depicts some embodiments of the deployed state of an occlusion device according to the present disclosure. In the deployed state, occlusion device 120 comprises a stem 121 and a flat spiral 122. Stem 121 may be an essentially straight wire segment, whereas flat spiral 122 occupies the shape of a flat disk.

Occlusion device 10 may be configured to be relatively stiff or, in some embodiments, relatively flexible. Alternatively, occlusion device 10 may be configured to assume any degree of flexibility. The typical distance δ between consecutive windings of flat spiral 122 is sufficiently small as to enable spiral 122 to quickly and efficiently become covered with endothelial cells. For example, the distance δ may be less than 1 mm, and, more specifically, less than 0.5 mm, and even more specifically, less than 0.2 mm.

Cavity occlusion device 120, according to some embodiments of the present disclosure, may be configured as a solid filament. Alternatively, it may be configured as a tube having a hollow lumen, or as a tube having its ends closed-off, thereby leaving an elongated air-space inside cavity occlusion device 120. Leaving an air-space inside cavity occlusion device 120 may have the advantage of making cavity occlusion device 120 more echogenic and therefore more highly visible by ultrasound imaging. Cavity occlusion device 10 may possess an echogenic marker or a radiopaque marker. Cavity occlusion device 120 may be made out the same materials as those indicated above for occlusion devices 10 and 11. Cavity occlusion device 120 may comprise an anchor (not shown) at the proximal end of stem 121.

Reference is now made to FIG. 12A, which depicts a body cavity 130 to be sealed. Body cavity 130, which may be, for example, a left atrial appendage or an aneurysm sac, comprises wall 131 and neck 132. Prior to sealing, fluid may freely communicate across the neck 132 of cavity 130.

Reference is now made to FIG. 12B, which depicts a side view of cavity occlusion device 120 implanted in cavity 130. The proximal end of stem 121 may protrude externally to wall 131 of cavity 130, thereby anchoring cavity occlusion device 120 in place. Whenever the proximal end of stem 121 possesses an anchor, further securement of cavity occlusion device 120 will take place. The proximal end of stem 121 may also be located in the wall 131 of cavity 130, or inside the interior 134 of cavity 130.

Flat spiral 122 of cavity occlusion device 120 is located across the neck 132 of cavity 130. The small distance δ between consecutive windings assures that endothelial cells from the vicinity of flat spiral 122 will deposit on the spiral and eventually create a contiguous tissue layer on the spiral. As a result, fluid communication across neck 132 will become impossible, and cavity 130 will be sealed and secured. If, for example, cavity 130 contains blood then placement of cavity occlusion device 120 in cavity 130 will cause blood inside interior 134 of cavity 130 to form clot 133.

It is noted that in some embodiments, cavity occlusion devices with more than one spiral are possible. Two or more flat disc spirals parallel to each other are possible. Spirals not parallel to each other are possible. A stem portion that is not straight, and has, for example, the geometry of a tangle, a skein, a bird's nest, or a cylindrical coil, is also possible.

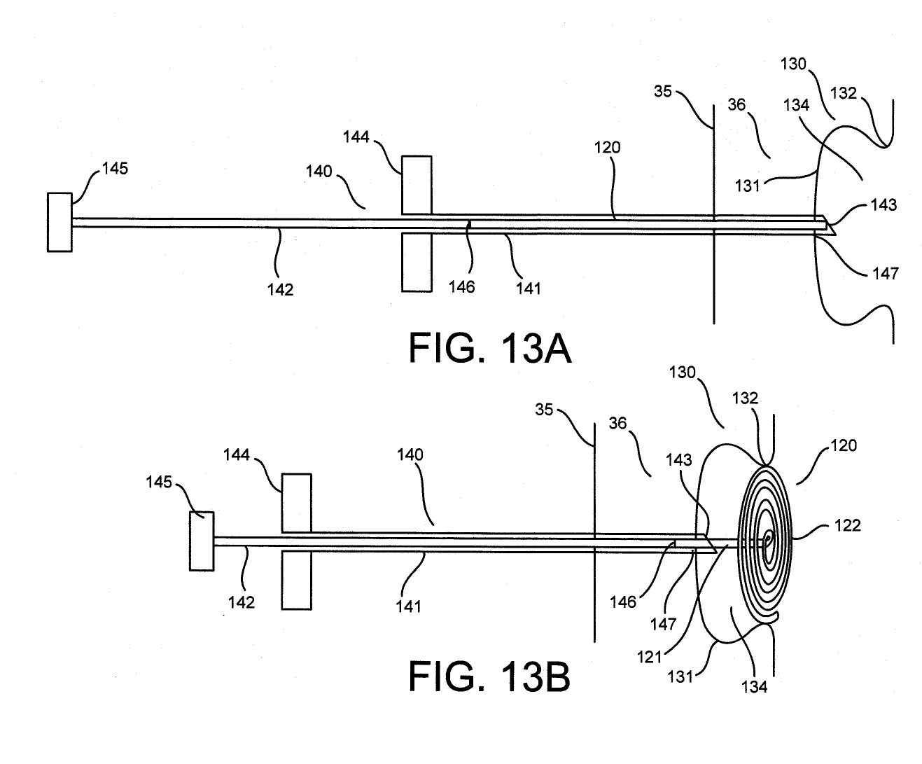

Reference is now made to FIGS. 13A-13B, which describe an apparatus and a method according to some embodiments of the present disclosure for implanting a cavity occlusion device according to some embodiments of the present disclosure. FIG. 13A depicts a delivery device 140 configured to implant cavity occlusion device 120 in body cavity 130. Delivery device 140 comprises a hollow needle 141, a pusher 142, and cavity occlusion device 120. Hollow needle 141 has a sharp end 143 configured to pierce skin 35, subcutaneous tissue 36, and the wall 131 of body cavity 130. Needle 141 may have a needle handle 144 located at its proximal end. The needle handle 144 may be rigidly connected to needle 141. Pusher 142 may have a pusher handle 145 located at its proximal end.

Hollow needle 141 may have a very small inner and outer diameter. For example, if the maximal collapsed diameter of undeployed stent 90 is 200 microns, the inner diameter of hollow needle 141 may be in the range of 200-600 microns, and the outer diameter of hollow needle 141 may be in the range of 300-800 microns. Thus, the puncture holes made by hollow needle 141 in a patient's tissue may be sufficiently small (300-800 microns) as to be self-sealing.

Hollow needle 141 may be made out of any suitable biocompatible material, such as, for example, steel. Pusher 142 may also be made out of a metal such as steel. Handles 144 and 145 may be made out of plastic.

Both cavity occlusion device 120 and pusher 142 are slidable within the lumen of hollow needle 141. Prior to deployment, cavity occlusion device 120 is located inside the lumen of needle 141 near its distal end 143. The distal end 146 of pusher 142 is also located inside the lumen of hollow needle 141. The distal end 146 of pusher 142 is in contact with the proximal end of cavity occlusion device 120. After deployment, as depicted in FIG. 13B, cavity exclusion device 120 is exteriorized from hollow needle 141, and the distal end of pusher 142 roughly coincides with distal end 143 of hollow needle 141.

The implantation of cavity occlusion device 120 in body cavity 130 may proceed as follows: First, an operator determines that it is desirable to implant cavity occlusion device 120 in body cavity 130. Under the guidance of a suitable imaging modality (not shown), such as, for example, ultrasound, high resolution ultrasound, angiography, CT scanning, any combination thereof, or without imaging guidance at all, the operator punctures skin 35 adjacent body cavity 130 using the sharp end 143 of needle 141. Note that delivery device 140 is in the configuration depicted in FIG. 13A, that is, with cavity occlusion device 120 housed near the distal end of hollow needle 141, in its undeployed, linear, substantially-straight wire state. The operator then carefully advances delivery device 140 through the subcutaneous tissue 36 (or between, for example, the ribs [not shown] of the patient if the cavity is a left atrial appendage), and punctures wall 131 of cavity 130 at puncture site 147. The tip 143 of needle 141 slightly protrudes into interior 134 of cavity 130. This situation is depicted in FIG. 13A.

Next, the operator holds needle 141 substantially motionless while advancing pusher 142 towards the patient. This may be done with one hand: the thumb of the operator pushes on pusher handle 145, whereas one or more fingers grasp needle handle 144. Thus, the distal end of cavity occlusion device 120 is advanced into interior 134 of cavity 130. As cavity occlusion device 120 is exteriorized from needle 141, it assumes its deployed shape of FIG. 12B, thereby sealing cavity 130 as in FIG. 12B. Generally, cavity occlusion device 120 will touch the wall 131 of cavity 130 at a location in the vicinity of neck 132. This situation is depicted in FIG. 13B.

It is noted that all absolute and relative motions of pusher 142 may be made using an automated mechanism, such as, for example, an automated electro-mechanical mechanism (not shown).

Once spiral 122 is suitably located in neck 132, and the proximal end of stem 121 is correctly located (for example, slightly protruding exteriorly from cavity 130), the operator extracts both needle 141 and pusher 142 from the patient's body. The implantation of cavity occlusion device 120 is complete.

In some embodiments, it is emphasized that cavity occlusion device 120, taken together with its delivery means 140, share the following characteristics: (i) The puncture hole made by delivery device 100 is sub-millimetric, and is therefore self-sealing and self-healing; (ii) The implant (that is, the cavity occlusion device) assumes the form of a substantially linear, substantially straight wire (monofilament) when in its undeployed state; (iii) The implant is implanted in the immediate vicinity of the cavity puncture site.

It is noted that embolic protection devices described in U.S. Provisional Patent Applications Nos. 61/653,676 and 61/693,979 to Shinar and Yodfat, incorporated herein by reference, at least in some embodiments, also share the following characteristics: (i) The puncture holes made by them are sub-millimetric, and are therefore self-sealing and self-healing; (ii) The implant (that is, the embolic protection device) assumes the form of substantially linear, substantially straight wire (monofilament) (such as, for example, a stretched helix) when in its undeployed state; (iii) The implant is implanted in the immediate vicinity of the cavity puncture site.

Although the embodiments of the present disclosure have been herein shown and described in what is conceived to be the most practical way, it is recognized that departures may be made from one and/or another of the disclosed embodiments and are within the scope of the present disclosure, which is not to be limited to the details described herein. The following exemplary claims aid in illustrating an exemplary scope of at least some of the embodiments disclosed herein.

1. A method for implanting a spatially bent and/or twisted implant in a patient, the method comprising:
providing a mono-filament implant configured to assume an undeployed, linear or linear-like state and a spatially bent and/or twisted deployed state, said implant having a proximal and a distal end;
creating a puncture in a vessel of said patient;
positioning said distal end of said implant in a lumen of a hollow needle such that said implant in the undeployed state substantially corresponds in shape to said lumen;
converting said implant from the undeployed state to the deployed state such that the proximal end of said implant is proximate said puncture.

2. The method of claim 1, wherein said implant:
is an occlusion device;
is a delivery platform for a therapeutic agent;
is a stent;
is a cavity occlusion device;
is an embolic protection device;
comprises at least one anchor; or
in the deployed state assumes the shape of a spiral, a helix, a bird's nest, or a skein.

3-8. (canceled)

9. The method of claim 1, further comprising loading said mono-filament implant in the undeployed state into a delivery catheter prior to the positioning said delivery catheter comprising a hollow needle of less than about 1 mm in diameter.

10. The method of claim 9, wherein converting comprises pushing said mono-filament implant out of said delivery catheter

11. (canceled)

12. A system for bringing about at least one of a vessel occlusion, a vessel ligation, a left atrial appendage occlusion, a patent foramen ovale occlusion, and opening or dilating stenosed or strictured vessels and maintaining vessel patency, comprising:
a mono-filament implant configured to assume an undeployed linear state and a spatially bent and/or twisted deployed state, and said implant having a proximal and a distal end,
wherein
upon positioning of said distal end of said implant in a lumen of a hollow needle, said implant is configured to substantially correspond in shape to said lumen in the undeployed state,
upon deployment, corresponds to said spatially bent and/or twisted deployed state, and
the proximal end of said implant is proximate a puncture in a vessel of a patient upon conversion of said implant from the undeployed state to the deployed state;
a delivery catheter comprising at least a hollow needle of less than about 1 mm in diameter, said needle having a lumen for housing said mono-filament implant in the undeployed state; and
a pusher configured to push said mono-filament implant from said delivery catheter.

13. A method for vessel ligation, comprising:
providing said system according to claim 12, wherein said implant further comprises a proximal anchor and a distal anchor;
puncturing a vessel wall at two diametrically-opposed sites;
retracting said needle away from said implant distal end allowing said distal anchor to self-expand,
optionally, further retracting said needle wherein said implant is exteriorized from said lumen,
upon said needle end being retracted to a point external to said lumen, said proximal anchor self-expands; and
sliding said proximal anchor towards said distal anchor, resulting in external compression of the vessel and adhering of the two opposing vessel walls.

14. The implant according to claim 11, wherein:
the bent and/or twisted state comprises a spiral that is formed by continuous winding with increasing or decreasing radius of curvature, or
said implant is covered by a swellable polymer that expands after contact with an aqueous environment

15. The implant according to claim 14, wherein:
the spiral is planar, and includes a concave or convex configuration; or
said implant comprises two spiral plates connected by a connecting neck.

16-17. (canceled)

18. (canceled)

19-20. (canceled)

21. A method for implanting a spatially bent and/or twisted implant in a patient, the method comprising:
providing a hollow needle having a lumen;
providing a monofilament implant configured to assume an undeployed, substantially linear state and a spatially bent and/or twisted deployed state, said implant having a proximal and a distal end;
placing said implant in said lumen of said needle in the undeployed state;
creating a puncture in a vessel of a patient;
positioning said needle in said vessel through said puncture; and
exteriorizing said implant from said lumen of said needle, thereby converting said implant from the undeployed state to the deployed state such that the proximal end of said implant is proximate said puncture.

22. The method of claim 21, wherein said needle has a sharp tip.

23. The method of claim 22, wherein the puncture is created using said sharp tip of said needle.

24. The method according to claim 21, wherein said needle has an outer diameter less than about 1 mm.

25. The method according claim 21 wherein said implant is one of: an occlusion device, a delivery platform for a therapeutic agent, a stent, a cavity occlusion device, and an embolic protection device.

26. The method according to claim 21, wherein said implant comprises at least one anchor.

27. The method according to claim 21, wherein said implant in the deployed state assumes the shape of a spiral, a helix, a bird's nest, or a skein.

28. The method according to claim 21, wherein said proximal end of said implant in the deployed state is proximal said puncture.

29. The method according to claim 21, wherein said needle is rigid.
